(19) United States
(12) Patent Application Publication
(10) Pub. No.: US 2025/0009431 A1
(43) Pub. Date: Jan. 9, 2025

(54) SYSTEM AND METHOD FOR DETERMINING FEMORAL CONTACT POINTS

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Ashley A. Roakes, Memphis, TN (US); Russell J. Brooke, Memphis, TN (US); Kara McCluskey, Memphis, TN (US); Mouhsin Ahmad El-Chafei, Arlington, TN (US)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 18/708,264

(22) PCT Filed: Oct. 7, 2022
(86) PCT No.: PCT/US22/46057

§ 371 Date: May 8, 2024

Related U.S. Application Data
(60) Provisional application No. 63/282,734, filed on Nov. 24, 2021.
(60) Provisional application No. 63/305,007, filed on Jan. 31, 2022.

(51) Int. Cl. A61B 34/10; A61F 2/46; G16H 20/40; G16H 50/70

(52) U.S. Cl. CPC A61B 34/10; A61F 2/46; A61F 2/4607; G16H 20/40; G16H 50/70; A61B 2034/104; A61B 2034/105; A61F 2002/4633

(57) ABSTRACT

Disclosed herein is a system and method for providing pre-operative planning and intra-operative guidance to a surgeon to assist in the optimal placement of the stem portion of a femoral implant within the intramedullary canal of a patient's femur during hip replacement surgery. In particular, the system and method provide information regarding contact points between the femoral stem in the patient's intramedullary canal to alleviate risk, inform the surgeon on key pre- and intra-operative decisions and guide femoral broaching and/or stem placement for optimal proximal fixation of the femoral implant.

### RELATED APPLICATIONS

This application claims the benefit of U.S. Provisional Applications Nos. 63/282,734, filed Nov. 24, 2021, and 63/305,007, filed Jan. 31, 2022, both entitled “System and Method for Determining Femoral Contact Points”, the contents of which are incorporated herein in their entireties.

### FIELD OF THE DISCLOSURE

The present disclosure relates generally to orthopedic devices and procedures and, more particularly, to a femoral hip stem implant procedure which includes systems and methods for assisting the surgeon to optimize the fit of the femoral implant within the femur of a patient.

### BACKGROUND

Orthopedic implants are well known and commonplace in today's society. Orthopedic implants may be used, for example, to stabilize an injury, to support a bone fracture, to fuse a joint, and/or to correct a deformity. Orthopedic implants may be attached permanently or temporarily and may be attached to the bone at various locations, including being implanted within a canal or other cavity of the bone, implanted beneath soft tissue and attached to an exterior surface of the bone, or disposed externally and attached by fasteners such as screws, pins, and/or wires. Some orthopedic implants allow the position and/or orientation of two or more bone pieces, or two or more bones, to be adjusted relative to one another.

One such orthopedic implant is a femoral hip stem implant used to repair or replace a hip joint. The hip joint is a frequent place for joint damage and/or injury. Femoral hip stem implants can be implanted or otherwise associated with the bony anatomy for treating traumatic injuries, reconstructing joint function, or for other purposes. Femoral hip stem implants may include an elongated insertion portion, referred to herein as the “stem portion”, which can be at least partially inserted into the intramedullary canal of the patient's proximal femur.

In some instances, the success of the femoral hip stem implant is dependent on how well the stem portion fits into the patient's bony anatomy (e.g., the intramedullary canal of the patient's femur). For example, it is important that proximal portions of the stem portion of the femoral implant fit tightly into the intramedullary canal, such that the implant loads proximal portions of the patient's femur, preventing bone loss through stress shielding and/or resorption (and potentially subsequent failure of the implant). It is also important that distal portions of the implant fit snugly into the intramedullary canal, however, the fit should not be so tight as to prevent proximal loading.

A good fit between the femoral implant and its associated bony anatomy may also help to prevent or lessen micromotion between the implant and the bone. Excessive micromotion may also lead to implant failure.

One major cause of femoral implant failure is poor osteointegration between the femoral implant and the intramedullary canal, often leading to the need for revision surgery. Achieving proximal fit is critical to the performance of the femoral implant. Currently, surgeons determine the size and fit of the femoral implant intra-operatively based on the best fit broach during femur preparation. After broaching, the surgeon will impact the femoral implant into place and assess the “fit and fill” of the femoral implant based on auditory cues or resistance felt during impaction of the femoral implant into the bone, often paired with a pre-operative assessment based on X-ray or other types of imaging. The final femoral broach position and the position of the femoral implant are expected to seat at the same level. However, in some cases, the femoral implant will sit “too proud” above the resection level or “too low” below the resection level.

Current pre-operative planning applications and tools do not display the points of contact between the femoral implant and the intramedullary canal of the femur. Thus, it would be beneficial to provide a tool, implemented as the system and method disclosed herein, to aid surgeons to visualize and understand the relationship between the femoral intramedullary canal and the femoral broaches and femoral implant, both pre-operatively and intra-operatively.

### SUMMARY OF THE DISCLOSURE

Disclosed herein is a system and method for providing information to a surgeon, both pre-operatively and intra-operatively, regarding the placement of the stem portion of a femoral implant within the intramedullary canal of a patient's femur during hip replacement surgery. In particular, the system and method provide information regarding points of contact between the stem portion of the femoral implant and the patient's intramedullary canal to alleviate risk, inform the surgeon on key pre- and intra-operative decisions and guide femoral broaching and/or stem placement for optimal proximal fixation of the femoral implant during a navigated surgical procedure.

In a pre-operative phase, the system and method uses patient imaging data, of the patient to build a 3-dimensional model of the patient's femur, to classify the femur and to make recommendations regarding the proper placement of the implant. Recommendations for a preferred surgical approach may also be made. Lastly, an assessment regarding the risk of complications based on the pre-operative plan are identified.

In an intra-operative phase, the system and method integrates with a navigated or robotic-assisted surgical platform. The system and method uses the pre-operative plan to assess the placement of the femoral implant within the intramedullary canal and allows for adjustment of the pre-operative plan intra-operatively. During a navigated procedure, the system and method may make recommendations regarding the broaching process to achieve optimal fit of the femoral implant to minimize risk of complications.

In one example, a method for providing a pre-operative plan for performing total hip arthroplasty includes building a 3D model of a femur from imaging data, performing auto-landmarking and measurement extraction on the model, classifying the femur based on the measured model, applying an algorithm to detect contact points between a stem of a femoral implant and an intramedullary canal of the femur and providing the pre-operative plan.

In any preceding or subsequent example, the method further includes providing pre-operative recommendations.

In any preceding or subsequent example, the method further includes wherein the pre-operative recommendations include recommendations regarding choosing an implant product, placement of the implant product and recommendations for a surgical approach.

In any preceding or subsequent example, the method further includes identifies risks of complications.

In any preceding or subsequent example, the method further includes wherein the identification of the risks of complications is informed by outcomes of procedures performed on patients having similar femur models and similar demographic and ancestry data.

In any preceding or subsequent example, the method further includes wherein the outcomes of previous procedures is stored in a database of 3D bone models.

In any preceding or subsequent example, the method further includes wherein the 3D bone models are grouped in the database by ancestry and demographic information of the various patients.

In any preceding or subsequent example, the method further includes searching the database for a model having similar demographic and ancestry dated to a patient to select a model to inform the pre-operative planning process.

In any preceding or subsequent example, the method further includes wherein the pre-operative plan is used as input for a navigated and/or robotic-assisted surgical procedure.

In any preceding or subsequent example, the method further includes collecting anatomical points during the navigated surgical procedure.

In any preceding or subsequent example, the method further includes providing an intra-operative plan displaying various viewpoints of the model of the femur with the intramedullary canal outlined.

In any preceding or subsequent example, the method further includes wherein the display of the model of the femur includes an overlay of the implant identifying the contact points between the implant and the intramedullary canal and identifying risks of complications.

In any preceding or subsequent example, the method further includes revising the pre-operative plan may be revised intra-operatively.

In any preceding or subsequent example, the method further includes tracking a spatial position of a femoral broach and displaying the track position of the femoral broach in the user interface.

In any preceding or subsequent example, the method further includes providing recommendations for positioning the femoral broach, to achieve optimal implant fit and implant seating for minimizing risks of complications.

In any preceding or subsequent example, the method further includes guiding removal of the femoral broach and guiding placement of the femoral implant.

In any preceding or subsequent example, the method further includes wherein a surgical outcome is stored in the database of 3D bone models, along with the model of the patient's femur and the patient's demographic and ancestry data.

In any preceding or subsequent example, the method further includes providing a graphical user interface is provided to assist a surgeon in the development of pre-operative plan.

In any preceding or subsequent example, the method further includes providing a graphical user interface to intra-operatively assist and guide the surgeon in the placement of the femoral implant to achieve an optimal fit.

In a second example, a system for performing a navigated and/or robotic-assisted surgical procedure includes a processor, a display and software that, when executed by the processor, causes the system to build a 3D model of a femur from imaging data, perform auto-landmarking and measurement extraction on the model, classify the femur based on the measured model, apply an algorithm to detect contact points between a stem of a femoral implant and an intramedullary canal of the femur and provide the pre-operative plan.

In any preceding or subsequent example, the system further includes a database of 3D bone models, wherein the software causes the system to perform the further functions of providing pre-operative recommendations regarding choosing an implant product, placement of the implant product and recommendations for a surgical approach and identifying risks of complications informed by outcomes of procedures performed on patients having similar femur models and similar demographic and ancestry data stored in the database.

In any preceding or subsequent example, the system further includes wherein the software causes the system to perform the further functions of displaying, on the display, the model of the femur including an overlay of the implant identifying the contact points between the implant and the intramedullary canal.

In any preceding or subsequent example, the system further includes a tracking device for spatially tracking an array of markers attached to a surgical instrument, wherein the software causes the system to perform the further functions of tracking a spatial position of the femoral broach, displaying the tracked position of the femoral broach in the display, providing recommendations for positioning the femoral broach to achieve optimal implant fit and implant seating for minimizing risks of complications, guiding removal of the femoral broach and guiding placement of the femoral implant.

Examples of the present disclosure provide numerous advantages. For example, the disclosed system and method provides insights and feedback that allow for improved efficiency in the operating room by reducing the number of broaches and trial components needed intra-operatively and therefore reducing the number of trays to be stored and sterilized for each case. In addition, the system and method provide the advantage of an intra-operative risk analysis that reduces the overall risk of post-operative complications and the need for revision surgery. Further, the disclosed system and method guides the use of new surgical instruments at key steps in the procedure to reduce the risk of intra-operative complications.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, a specific exemplary example of the disclosed system and method will now be described, with reference to the accompanying drawings, in which:

FIG. 1 is a flowchart showing a workflow of a first example of the present disclosure providing a pre-operative planning tool;

FIG. 2 is a flowchart showing a workflow of a second example of the present disclosure providing an intra-operative planning and monitoring tool;

FIG. 3 illustrates the landmarks and measurements on the femur of a patient that are automatically determined by various examples of the present disclosure.

FIG. 4 is an exemplary user interface screen from the pre-operative portion of the system of the present disclosure.

FIG. 5 is an exemplary user interface screen from the intra-operative portion of the system of the present disclosure.

FIG. 6 depicts an operating theatre including an illustrative computer-assisted surgical system with which various examples of the present disclosure could be used.

### DETAILED DESCRIPTION

The system and method disclosed herein provides information to a surgeon during the pre-operative and intra-operative phases of the hip replacement surgery which will assist the surgeon in choosing the proper model and size of the femoral implant, the optimal placement of the femoral implant and the surgical approach, based on a match between the geometry of the patient's intramedullary canal and the geometry of the chosen implant. In particular, the system and method visualizes the placement of the stem of the femoral implant in the intramedullary canal and identifies the points of contact between the femoral implant and the intramedullary canal.

Pre-operatively, the disclosed system in method can provide information that can be used to determine the implant size, to guide femoral broaching, for example, to determine the orientation of the broach, skip intermediate broach sizes, etc., and assess the risk of fracture or subsidence prior to surgery. In addition, a pre-operative plan is generated.

Intra-operatively, the disclosed system and method can guide the surgeon in the broaching procedure and update the pre-operative plan based on the actions of the surgeon during femoral broaching. For example, the system and method may inform the surgeon of updated contact points, output risk assessment, adjust positions of the implant based on the broach trajectory, etc.

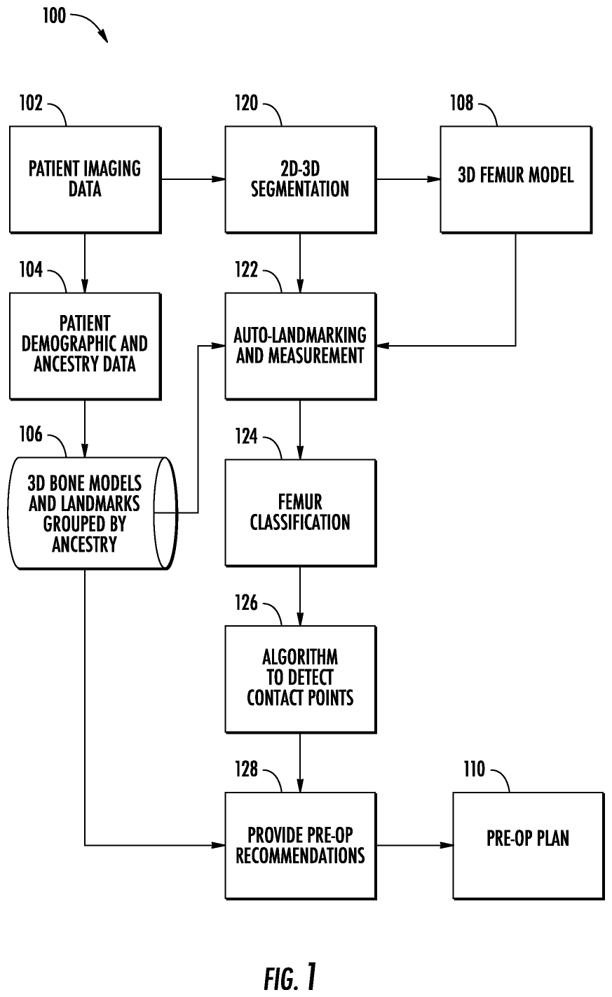

FIG. 1 is a flowchart showing an exemplary workflow 100 of a first example of the disclosed system and method implementing the pre-operative phase.

An input to the pre-operative phase of the system and method is patient DICOM data 102. (DICOM is an international standard to transmit, store, retrieve, print, process, and display medical imaging information). DICOM data may represent, for example, X-ray imaging, CT imaging, MRI imaging, etc., which is collected pre-operatively. The DICOM data may have metadata associated therewith including patient demographic and ancestry data 104. Alternatively, the demographic and ancestry data of the patient 104 may be collected through other means, for example, by interviewing the patient.

Demographic and ancestry data of the patient 104 may be stored in a database 106 including 3D bone models and landmarks grouped by ancestry.

The DICOM imaging data is used to perform a 2D-3D segmentation at 120 to generate a 3D model of the patient's femur from the 2D imaging data. Preferably the DICOM data will include either CT or MRI imaging data. Software implementing the 2D-3D segmentation process takes 2D slices from the DICOM imagery and forms a 3D model of the patient's femur. The 2D-3D segmentation process 120 outputs a 3D model of the patient's femur 108.

It is known that there are patterns between the femoral models generated for patients belonging to common demographic and ancestral groups. Therefore, in some examples, the 3D bone model and landmarks database 106 may contain a femoral model similar to the model 108 of the patient's femur as indicated by the patient demographic and ancestry data 104. A closely-matching femoral model may be identified by similarities between the patient's demographic and ancestry data 104 and the demographic and ancestral data associated with the femoral models stored in the database 106. The identified model from database 106 may have information associated therewith regarding, inter alia, locations of landmarks, the size and model of the implant used with the femoral model, risks identified during surgery in which the model was used, the surgical approach used during surgery in which the model was used, etc. In addition, the identified model may have outcome information from a previous procedure in which the model was used, noting problems or complications encountered during the procedure, as well as the ultimate outcome of the procedure, such as to inform the risk assessment process for the current procedure.

During process 122, landmarks and measurements of the intramedullary canal and cortical capsule of the patient are obtained from the 3D femoral model 108 of the patient. In some examples, in process 122, the full femur model is sliced axially along the diaphyseal region of the femur. From the diaphyseal cross-sections, the cortical thickness and intramedullary (IM) diameters can be calculated. The cross-section of the IM canal that has the smallest inner canal diameter is identified as the isthmus. Once the isthmus location is found, the proximal femoral axis is identified as a line fit to the center points of all the cross-sections superior to the isthmus. The femoral head center is calculated by fitting a sphere to the most medial and proximal portion of the femur through a stochastic point sampling process. A proximal region of the femur, below the femoral head center and above the shaft isthmus, is sliced axially to obtain the cross-sections for identifying the lesser trochanter. The lesser trochanter peak can be approximated by detecting the changes in the ML width between each cross section. The femoral neck axis can be approximated as the line connecting the femoral head center and a point indicated by the position of the lesser trochanter. Cross-sections taken orthogonally to the approximated neck axis can identify the femoral neck isthmus location and diameter. These landmarks can be used to calculate the following femoral measurements: femoral head diameter, femoral head offset, neck shaft angle, femoral head height, isthmus height, anteversion angle, neck length, isthmus AP and ML dimensions, etc. The full femur model is sliced axially along the diaphyseal region of the femur. From the diaphyseal cross-sections, the cortical thickness and intramedullary (IM) diameters can be calculated. The cross-section of the IM canal that has the smallest inner canal diameter is identified as the isthmus. Once the isthmus location is found, the proximal femoral axis is identified as a line fit to the center points of all the cross-sections superior to the isthmus. The femoral head center is calculated by fitting a sphere to the most medial and proximal portion of the femur through a stochastic point sampling process. A proximal region of the femur, below the femoral head center and above the shaft isthmus, is sliced axially to obtain the cross-sections for identifying the lesser trochanter. The lesser trochanter peak can be approximated by detecting the changes in the ML width between each cross section. The femoral neck axis can be approximated as the line connecting the femoral head center and a point indicated by the position of the lesser trochanter. Cross-sections taken orthogonally to the approximated neck axis can identify the femoral neck isthmus location and diameter. These landmarks can be used to calculate the following femoral measurements: femoral head diameter, femoral head offset, neck shaft angle, femoral head height, isthmus height, anteversion angle, neck length, isthmus AP and ML dimensions, etc. In other examples, other processes may be used.

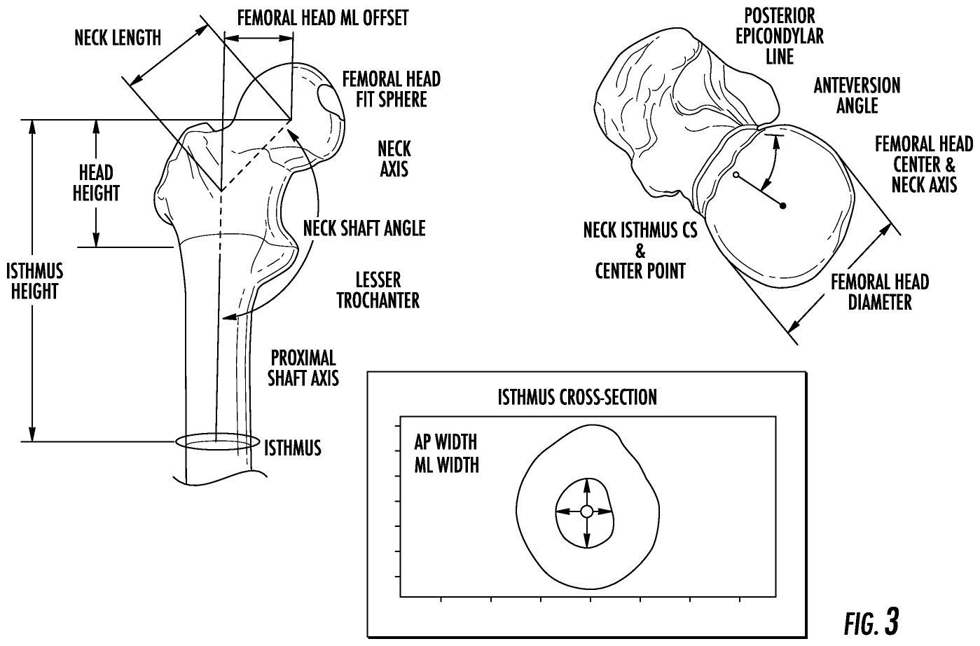

FIG. 3 shows exemplary landmarks and measurements of a typical femur which may be identified and collected by process 122. The information associated with the identified femoral model from database 106 may be used as input to inform or supplement the process of auto-landmarking and measurement extraction process 122 for the patient. In some examples, a factor analysis can be performed on similar models identified in database 106 to define commonalities between the model 108 of the patient's femur and similar models from database 106. If common features are found between the model 108 of the patient's femur and similar model in database 106, they are used as discussed later herein.

In the event no matching or closely-matching femoral model is found in database 106, the process may continue based solely on the patient's 3D femoral model 108. In either case, the patient's 3D femoral model 108, as well as associated metadata and information from and the outcome of the surgical procedure may be added to database 106 for use in future procedures.

Auto-landmarking process 122 may, in some examples, use machine learning modules. A machine learning model may take, for example, the 3D model 108 of the patient's femur and, based on a coordinate system of the CT or MRI apparatus, which was used to obtain the DICOM imagery, the machine learning model may look for specific patterns within the model 108 to identify the landmarks for that case.

Based on the results of the auto-landmarking and measurement extraction process 122, classification process 124 performs a DORR classification on the model of the patient's femur. The DORR classification is based on the shape and dimensions of the intramedullary canal and cortical thickness. The DORR standard is industry-standard classification wherein a femur is classified as type “A”, in which the femur has a narrow intramedullary canal with thick cortical walls, type “B”, having a moderately-sized intramedullary canal and moderate cortical walls or type “C”, in which the femur has a wide intramedullary canal with thin cortical walls.

Classification process 124 takes slices along the intramedullary canal from 3D model 108 and obtains the thickness of the cortical shell (i.e., the distance between the outer surface 402 and the inner surface 404 of the bone) and provides a DORR classification, classifying the femur as type A, B, or C, based on canal shape. The DORR classification informs the selection of the type and size of the femoral implant that will be chosen for implantation.

Once the classification, the shape of the canal, and measurements for the canal are known, a specific implant may be selected. Algorithm 126 detects contact points between the stem of the chosen femoral implant and the intramedullary canal (from the chosen femoral stem). In some examples, algorithm 126 outputs the contact points based on the boundary of the patient's intramedullary canal, proximal characteristics of the femur, selected implant geometry, and the approximated trajectory of the femoral canal. This is achieved by using an implant library that contains the geometry for each femoral stem option. The chosen femoral stem geometry is overlaid with the geometry of the patient's intramedullary canal and aligned to the femoral shaft axis which is representative of the alignment of the femoral stem in the IM canal. The contact points are identified as the regions where the implant and femur model overlap. In an alternate example, contact points may be identified by having the algorithm virtually “implant” different components to optimize the fit between the implant and bone as described above. In yet other examples, other algorithms may be used.

In a display, the geometry from the model of the intramedullary canal may be overlaid with the geometry of the implant to identify where the stem of the chosen femoral implant will contact the walls of the intramedullary canal. In some examples, algorithm 126 may be implemented as a trained machine learning model trained to detect the contact points given the landmarks and measurements on the model 108 of the patient's femur and known types and sizes of femoral implants.

Process 128 provides pre-operative recommendations based on all known information, including, for example, the 3D femur model 108 of the patient identifying landmarks and measurements, the femur classification and the points of contact between the intramedullary canal and the chosen femoral implant. In some examples, during process 128, the intramedullary canal geometry and shape are used to identify the best stem design for that anatomy. The algorithm also detects the best size and position of the implant by virtually “implanting” the geometry of a variety of implants within the patient's proximal femur. The best design, size, and position (anteversion, lateralization, etc.) of the stem is determined by the combination that results in the most contact between the IM canal and the implant within boundaries of the IM canal based on surgical approach. In other examples, other methods may be used for process 128.

The preoperative recommendations may include, for example, recommendations for the implant product to be used based on measurements of intramedullary canal, recommendations on the placement and orientation of the femoral implant and surgical approach (i.e., posterior, lateral, anterior). Recommendation process 128 may also identify risks of intra-operative and post-operative complications based on outcomes from similar cases stored in database 106. For example, recommendation process 128 may determine a risk of subsidence of the femoral implant or a risk of the femoral implant sitting too proud.

In addition, recommendation process 128 may also predict intra-operative complications during the pre-operative planning process and prior to surgery and may advise on which stems would have the least risk of complications. For example, recommendation process 120 may be able to predict trochanteric fractures, femoral fractures or acetabular fractures. The predictions of recommendation process 128 may be based on identifying high-risk patients based on native anatomy obtained from medical imaging and patient demographic and ancestry data, which can be used to identify similar cases and their outcomes stored in database 106. Database 106, containing outcomes of previous surgical procedures may be used to advise on methods or approaches most likely to result in success given specific patient anatomy.

Recommendation process 128 may be informed by specific outcomes of similar models identified in the database 106, based on the patient's demographic and ancestry data 104. Database 106 contains cases that have been previously analyzed with algorithm 126, so the pre-operative plans and outcomes of those cases are known and can used by recommendation process 128 to formulate recommendations for the current case.

Pre-operative process 100 outputs a pre-operative plan 110, which may be used in the intra-operative phase of the disclosure, which will be discussed next. The method of process 100, involving the pre-operative planning, may be implemented, in some examples, as a system including a software application that can be executed on any computing device, for example, a laptop computer, a tablet computer, etc.

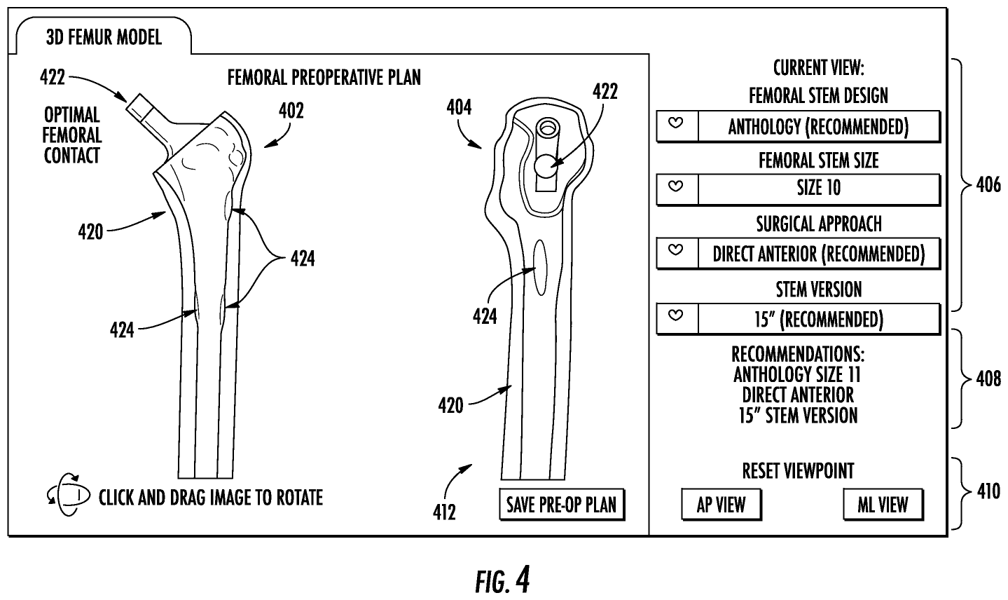

An exemplary interactive user interface screen for the software application for the pre-operative workflow 100 is shown in FIG. 4. In the user interface, there can be multiple viewpoints 402, 404 of the femur 420, the implanted femoral stem 422 and the contact regions 424 between the femur 420 and the implanted femoral stem 422. The surgeon can choose which stem design, stem size, planned surgical approach, and stem version to display in a model by using the selection tools 406. Additionally, the pre-operative planning tool makes recommendations 408 on the best stem design and size, surgical approach, and stem anteversion for optimal femoral contact. Further, because the interface allows the surgeon to manipulate and rotate the model views, 402, 404, the user can snap the view to specific viewpoints by selecting one of the buttons in area 410 of the user interface which may allow, for example, anterior posterior (“AP”, i.e., front to back) views or medial lateral (“ML”, i.e., side to side) views of the femur by selection of the appropriate button. Lastly, when the surgeon is satisfied with the pre-operative plan, the plan can be saved by a button selection 412. In the second example of the present disclosure, the pre-operative plan is saved as an input to the intra-operative phase of the present disclosure.

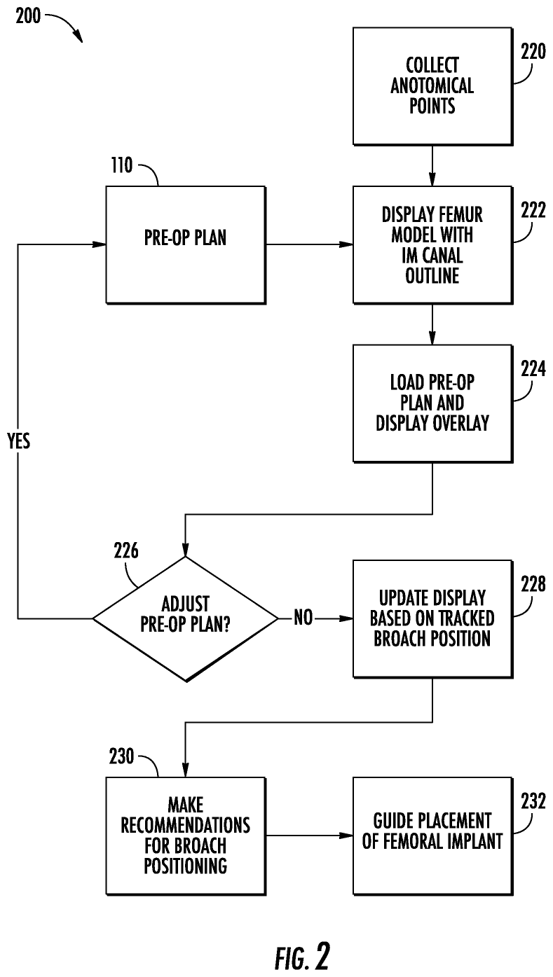

FIG. 2 is a flowchart showing an exemplary workflow 200 of a second example of the disclosed system and method implementing the intra-operative phase. In the second example of the present disclosure, the intra-operative phase workflow 200 is used in addition to the pre-operative workflow 100 previously discussed. The second example of the present disclosure is useful in instances where the hip replacement procedure is a navigated procedure or a robotically-assisted procedure.

The pre-operative plan 110, which is the output of workflow 100, is used as an input to the navigation/robotics software, which can refer to pre-operative plan 110 throughout the navigated procedure. The software of the navigated/robotic-assisted system can modify pre-operative plan 110 intra-operatively by understanding the spatial relationships femur of the patient and relating the spatial relationship to the model in pre-operative plan 110.

At process 220, anatomical points are intra-operatively identified and their spatial positionings with respect to an established coordinate system are collected during the navigated procedure. In some examples, during process 220, an intra-operative point collection is carried out using a navigated pointer probe to collect anatomical points that were previously identified on the pre-operative bone model. Examples of anatomical points could include collecting points for the acetabulum to approximate femoral head center, identifying the level of resection of the bone, the peak of the lesser trochanter, etc. In other examples, other methods may be used for process 220.

Based on the identified anatomical points, the 3D model of the patient's femur can be aligned to the software's statistical shape model of the intra-operative bone through a generalized Procrustes analysis. At process 222, a statistical shape model of the patient's femur is displayed with the intramedullary canal outlined in AP and ML views.

At 226, the pre-operative plan 110 is loaded and an overlay of the femoral implant, the contact points between the femoral implant in the intramedullary canal and the complication risk areas are displayed on the statistical shape model.

At 228 the surgeon determines, based on information presented in the display, whether pre-operative plan 110 provides the optimal contact as-is or whether the pre-operative plan 110 should be adjusted to achieve optimal contact between the femoral implant and the intramedullary canal. The surgeon may adjust pre-operative plan 110, in which case the display is updated to reflect the new pre-operative plan 110. The adjustments may include, for example, modifying the sizing of the stem, deciding whether the placement of the stem should be changed (i.e., more lateral or not), increasing the number of points of contact or to correct for something that occurred intra-operatively (e.g., a fracture). Any adjustments made to the pre-operative plan 110 are saved into database 106 and used to refine algorithm 126 from pre-operative workflow 100.

During the procedure, at 228, the navigation system may track the femoral broaching process to make sure the pre-operative or revised pre-operative plan 110 is being followed. The positions of instruments may be tracked with respect to specific markers to determine their spatial positioning. For example, the position of the broach may be tracked with sensors attached to the broach handle.

At process 230, the system may make recommendations to the surgeon to move the broach or modify the broaching process to achieve optimal implant fit, seating, contact points and minimizing the risk of complications. For example, recommendations may be made for lateralizing the broach or for up-sizing the broach. At 232, the broach is removed and implant is placed. In some examples, workflow 200 may include an option to guide placement of the implant.

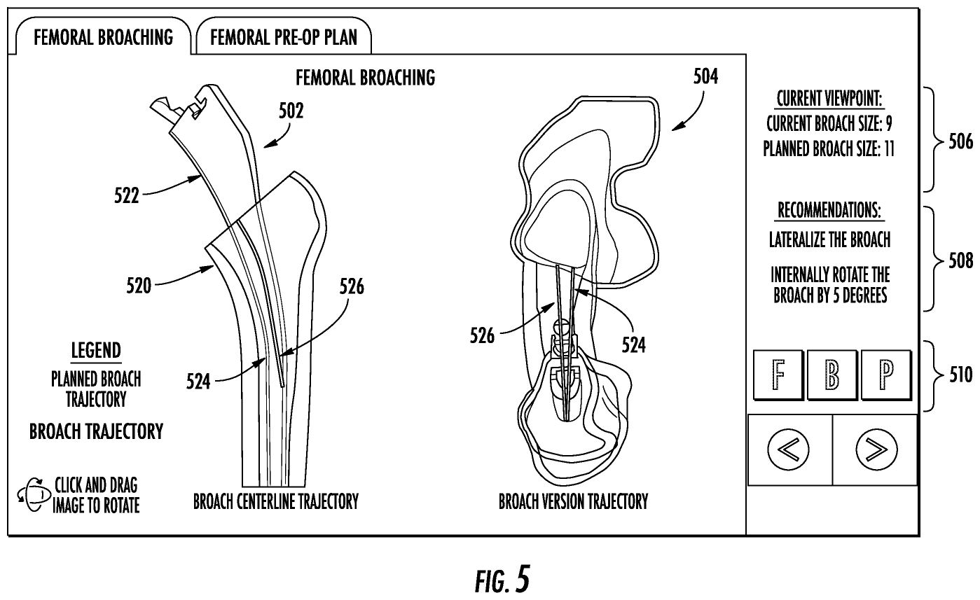

FIG. 5 shows an exemplary screen of the user interface of the system during the intra-operative phase, showing a navigation GUI during the femoral broaching step of the procedure. In the user interface, there can be multiple viewpoints 502, 504 of femur 520, the femoral broach 522 and the trajectories of the planned position 524 of the stem for optimal contact and the live position 526 of the broach that is being tracked through the navigation platform. An additional viewpoint that may be of interest is a view looking down the femoral canal (view 504) which is more likely to be the real view of the surgeon of the patient anatomy, depending on the surgical approach. This view could display the planned 524 and live 526 anteversions of the broach trajectory. Panel 506 may display the current and planned broach sizes. In addition to viewing the live position 526 of the broach and how it relates to the planned trajectories 524, the algorithm can output recommendations for achieving the surgical plan in the panel indicated by 508 and the recommendations can be live-updated as the surgeon broaches the femur. The status of the tracking arrays for navigation platform are displayed in the panel indicated by 510 wherein the selection of “F” displays the femur tracking array, the selection of “P” displays the pelvis tracking array and a selection of “B” displays the broach tracking array. These indicators may be displayed in red if a tracking array is not visible and needs to be adjusted.

The intra-operative workflow 200 may be implemented as software application running on a computing system interfaced with a navigated or robotic-assisted surgical platform or, alternatively, as part of the software of the navigated or robotic-assisted surgical platform.

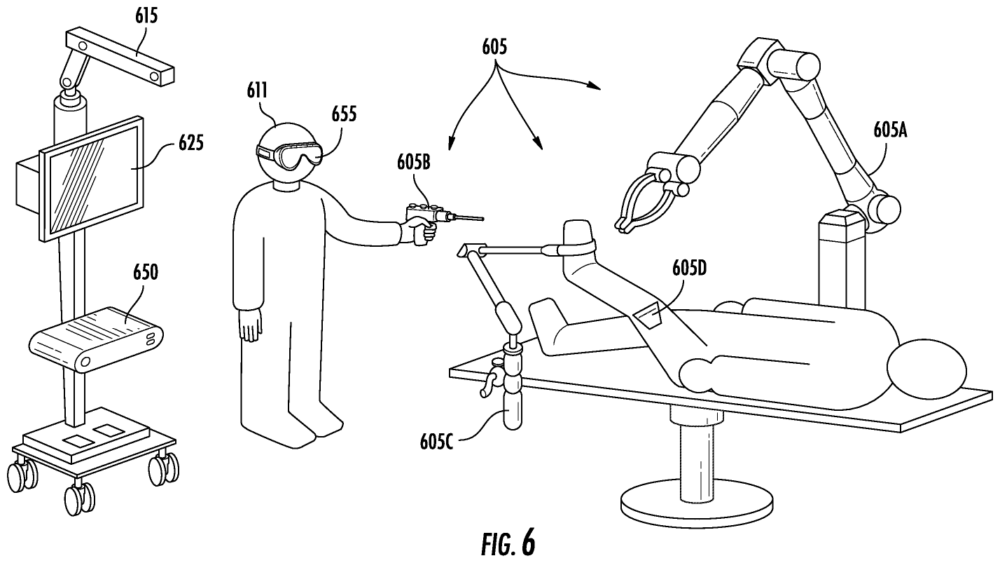

FIG. 6 provides an illustration of an exemplary computer-assisted surgical system 600, with which the system and method of the present disclosure may be implemented. System 600 uses computers, robotics, and imaging technology to aid surgeons in performing orthopedic surgery procedures such as total knee arthroplasty (TKA) or total hip arthroplasty (THA). For example, surgical navigation systems can aid surgeons in locating patient anatomical structures, guiding surgical instruments, and implanting medical devices with a high degree of accuracy. Surgical navigation systems 600 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An effector platform 605 positions surgical tools relative to a patient during surgery. The exact components of the effector platform 605 will vary, depending on the example employed. For example, for a knee surgery, the effector platform 605 may include an end effector 605B that holds surgical tools or instruments during their use. The end effector 605B may be a handheld device or instrument used by the surgeon (e.g., a NAVIO® hand piece or a cutting guide or jig) or, alternatively, the end effector 605B can include a device or instrument held or positioned by a robotic arm 605A.

The effector platform 605 can include a limb positioner 605C for positioning the patient's limbs during surgery. One example of a limb positioner 605C is the SMITH & NEPHEW SPIDER2 system. The limb positioner 605C may be operated manually by the surgeon or alternatively change limb positions based on instructions received from the surgical computer 650 (described below).

Resection equipment (e.g., end effector 605B in FIG. 6) performs bone or tissue resection using, for example, mechanical, ultrasonic, or laser techniques. Examples of resection equipment include drilling devices, burring devices, oscillatory sawing devices, vibratory impaction devices, reamers, ultrasonic bone cutting devices, radio frequency ablation devices, and laser ablation systems. In some examples, the resection equipment is held and operated by the surgeon during surgery. In other examples, the effector platform 605 may be used to hold the resection equipment during use.

The effector platform 605 can also include a cutting guide or jig 605D that is used to guide saws or drills used to resect tissue during surgery. Such cutting guides 605D can be formed integrally as part of the effector platform 605 or robotic arm 605A or cutting guides can be separate structures that can be removably attached to the effector platform 605 or robotic arm 605A. The effector platform 605 or robotic arm 605A can be controlled by the system 600 to position a cutting guide or jig 605D adjacent to the patient's anatomy in accordance with a pre-operatively or intra-operatively developed surgical plan such that the cutting guide or jig will produce a precise bone cut in accordance with the surgical plan.

The tracking system 615 uses one or more sensors to collect real-time position data that locates the patient's anatomy and surgical instruments. For example, for TKA procedures, the tracking system 615 may provide a location and orientation of the end effector 605B during the procedure. In addition to positional data, data from the tracking system 615 can also be used to infer velocity/acceleration of anatomy/instrumentation, which can be used for tool control. In some examples, the tracking system 615 may use a tracker array attached to the end effector 605B to determine the location and orientation of the end effector 605B. The position of the end effector 605B may be inferred based on the position and orientation of the tracking system 615 and a known relationship in three-dimensional space between the tracking system 615 and the end effector 605B. Various types of tracking systems may be used in various examples of the present disclosure including, without limitation, Infrared (IR) tracking systems, electromagnetic (EM) tracking systems, video or image based tracking systems, and ultrasound registration and tracking systems.

The display 625 provides graphical user interfaces (GUIs) that display images the user interface of the navigated surgical platform, as well as the user interface of the systems and methods of the present disclosure, shown in FIG. 4 and FIG. 5, as well other information relevant to the surgery. For example, in some examples, the display 625 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The display 625 may include, for example, one or more computer monitors. As an alternative or supplement to the display 625, one or more members of the surgical staff may wear an augmented reality (AR) Head Mounted Device (HMD). For example, in FIG. 6 the surgeon 611 is wearing an AR HMD 655 that may, for example, overlay pre-operative image data on the patient or provide surgical planning suggestions. Various example uses of the AR HMD 655 in surgical procedures are detailed in the sections that follow.

Surgical computer 650 provides control instructions to various components of system 100, collects data from those components, and provides general processing for various data needed during surgery. In some examples, the surgical computer 650 is a general purpose computer. In other examples, the surgical computer 650 may be a parallel computing platform that uses multiple central processing units (CPUs) or graphics processing units (GPU) to perform processing. In some examples, the surgical computer 650 is connected to a remote server over one or more computer networks (e.g., the Internet). The remote server can be used, for example, for storage of data or execution of computationally intensive processing tasks.

In various examples, the intra-operative workflow 200 of the present disclosure may be implemented as a software application executing on surgical computer 650 integrated with or separate from the software controlling the navigated surgical platform. Also, in various examples, the pre-operative workflow 100 may be implemented as a software application executing on a computing platform other than surgical computer 650, such as a laptop or tablet computing device. The computing device executing the pre-operative workflow 100 may be in communication with surgical computer 650 such as to provide the pre-operative plan 110 to intra-operative workflow 200.

Various examples of a system and method have been described herein to provide a surgeon performing THA procedures with a tool for visualizing the placement of the femoral implant relative to the intramedullary canal and provide recommendations for selection of the implant size and type, the surgical approach, and the proper placement of the implant. In some examples a pre-operative tool was provided to develop a pre-operative plan including a plan for the optimal placement of a pre-chosen femoral implant and the identification of risks of complications. In a second example, the present disclosure is integrated with the navigated surgical platform to guide the surgeon during the procedure to achieve an optimal fit of the implant within the intramedullary canal.

System and method provides improved intra-operative efficiency by providing intra-operative insights and feedback regarding the contact points between the femoral implant and the intramedullary canal. The insights and feedback provided in the workflow could provide possibilities for the surgeon to skip broach sizes, reduce the need for trialing and/or assess the risk of the stem portion of the femoral implant sitting too proud or subsiding intra-operatively based on the patient's anatomy.

The system may also provide improved efficiency in the operating room. The pre-operative planning tool could help to reduce the number of broaches and trial components needed intra-operatively, thus reducing the number of trays to be stored and sterilized for each case.

Lastly, the intra-operative risk analysis could reduce the overall risk of post-operative complications and the need for revision surgery. In addition, the system and method of the present disclosure could guide the use of new surgical instruments at key steps in the procedure to reduce the risk of intra-operative complications.

We claim:

1. A method for providing a pre-operative plan for performing total hip arthroplasty comprising:
building a 3D model of a femur from imaging data;
performing auto-landmarking and measurement extraction on the model;
classifying the femur based on the measured model;
applying an algorithm to detect contact points between a stem of a femoral implant and an intramedullary canal of the femur; and
providing the pre-operative plan.

2. The method of claim 1, further comprising:
providing pre-operative recommendations.

3. The method of claim 2, wherein the pre-operative recommendations include recommendations regarding choosing an implant product, placement of the implant product and recommendations for a surgical approach.

4. The method of claim 1, further comprising:
identifies risks of complications.

5. The method of claim 4, wherein the identification of the risks of complications is informed by outcomes of procedures performed on patients having similar femur models and similar demographic and ancestry data.

6. The method of claim 5, wherein the outcomes of previous procedures is stored in a database of 3D bone models.

7. The method of claim 6, wherein the 3D bone models are grouped in the database by ancestry and demographic information of the various patients.

8. The method of claim 7, further comprising:
searching the database for a model having similar demographic and ancestry dated to a patient to select a model to inform the pre-operative planning process.

9. The method of claim 1, wherein the pre-operative plan is used as input for a navigated and/or robotic-assisted surgical procedure.

10. The method of claim 9, further comprising:
collecting anatomical points during the navigated surgical procedure.

11. The method of claim 1, further comprising:
providing an intra-operative plan displaying various viewpoints of the model of the femur with the intramedullary canal outlined.

12. The method of claim 11, wherein the display of the model of the femur includes an overlay of the implant identifying the contact points between the implant and the intramedullary canal and identifying risks of complications.

13. The method of claim 1, further comprising:
revising the pre-operative plan intra-operatively.

14. The method of claim 1, further comprising:
tracking a spatial position of a femoral broach; and
displaying the track position of the femoral broach in the user interface.

15. The method of claim 14, further comprising:
providing recommendations for positioning the femoral broach, to achieve optimal implant fit and implant seating for minimizing risks of complications.

16. The method of claim 15, further comprising:
guiding removal of the femoral broach; and
guiding placement of the femoral implant.

17. The method of claim 16, wherein a surgical outcome is stored in the database of 3D bone models, along with the model of the patient's femur and the patient's demographic and ancestry data.

18. The method of claim 1, further comprising:
providing a graphical user interface is provided to assist a surgeon in the development of pre-operative plan.

19. The method of claim 18, further comprising:
providing a graphical user interface to intra-operatively assist and guide the surgeon in the placement of the femoral implant to achieve an optimal fit.

20. A system for performing a navigated and/or robotic-assisted surgical procedure comprising:
a processor;
a display; and
software that, when executed by the processor, causes the system to:
build a 3D model of a femur from imaging data;
perform auto-landmarking and measurement extraction on the model;
classify the femur based on the measured model;
apply an algorithm to detect contact points between a stem of a femoral implant and an intramedullary canal of the femur; and
provide the pre-operative plan.

21. The system of claim 20, further comprising:
a database of 3D bone models;
wherein the software causes the system to perform the further functions of:
providing pre-operative recommendations regarding choosing an implant product, placement of the implant product and recommendations for a surgical approach; and
identifying risks of complications informed by outcomes of procedures performed on patients having similar femur models and similar demographic and ancestry data stored in the database.

22. The system of claim 20, wherein the software causes the system to perform the further functions of:
displaying, on the display, the model of the femur including an overlay of the implant identifying the contact points between the implant and the intramedullary canal.

23. The system of claim 20, further comprising:
a tracking device for spatially tracking an array of markers attached to a surgical instrument;
wherein the software causes the system to perform the further functions of:
tracking a spatial position of the femoral broach;
displaying the tracked position of the femoral broach in the display;
providing recommendations for positioning the femoral broach to achieve optimal implant fit and implant seating for minimizing risks of complications;
guiding removal of the femoral broach; and
guiding placement of the femoral implant.
